(19) United States
(12) Patent Application Publication
(10) Pub. No.: US 2014/0057840 A1
(43) Pub. Date: Feb. 27, 2014

(54) ANTI-ANDROGENIC AGENT, SEBUM SECRETION BLOCKER, HAIR GROWTH STIMULANT, AND FOOD OR BEVERAGE

(71) Applicant: LOTTE CO., LTD., Tokyo (JP)

(72) Inventors: Yushi Kodama, Sayama-shi (JP); Hiroaki Higuchi, Koshigaya-shi (JP); Atsushi Narise, Kawasaki-shi (JP); Koji Sakurai, Takasaki-shi (JP)

(73) Assignee: LOTTE CO., LTD., Tokyo (JP)

(21) Appl. No.: 14/070,513

(22) Filed: Nov. 2, 2013

Related U.S. Application Data
(62) Division of application No. 13/518,890, filed on Sep. 10, 2012.

(30) Foreign Application Priority Data
Dec. 24, 2009 (JP) 2009-292651

(51) Int. Cl. A61K 38/40; A23L 1/305

(52) U.S. Cl. CPC A61K 38/40; A23L 1/3056
USPC 514/5.4

(57) ABSTRACT

Provision of an anti-androgenic agent which has a strong anti-androgenic action, is free of side effects and very safe, and a sebum secretion blocker and a hair growth stimulant containing the anti-androgenic agent as an active ingredient. Provided are the anti-androgenic agent comprising lactoferrin, the sebum secretion blocker containing the anti-androgenic agent as an active ingredient, the hair growth stimulant containing the anti-androgenic agent as an active ingredient, and a food or drink product comprising the anti-androgenic agent containing lactoferrin as an active ingredient.

### TECHNICAL FIELD

The present invention relates to an anti-androgenic agent, and a sebum secretion blocker and a hair growth stimulant containing the anti-androgenic agent as an active ingredient.

### BACKGROUND ART

Most steroid hormones develop their actions by binding to a receptor in the molecular shape as secreted by a production organ, however, it is not the case with the male hormones collectively termed as androgen. For example, testosterone is produced in the hypophysis, adrenal gland, testis, and the like, and reduced to 5α-dihydrotestosterone (DHT) by testosterone 5α-reductase in the cells of a target organ before binding to the receptor and developing the action as androgen.

Androgen is an important class of hormone, but its excessive action induces various unfavorable conditions such as andromorphous baldness, trichauxis, seborrhiasis, acne, prostatomegaly, prostate gland tumor, premature development in boys, etc. Accordingly, it has been considered to improve these unfavorable conditions by suppressing the excessive androgen actions. To achieve it, there can be a method for blocking the action of testosterone 5α-reductase, which reduces testosterone to the active DHT, to suppress the production of the active DHT; and another method for preventing DHT produced from testosterone from binding to a receptor so as not to develop the action.

Consequently, this time, some of the natural materials were screened by carrying out testosterone 5α-reductase blocking test, androgen receptor binding blocking test and further sebaceous gland function suppression evaluation using cultured hamster sebocytes, and it has been found that lactoferrin present in various secretory fluids, such as milk, tears, saliva, bile, and the like, of mammals may have a strong anti-androgenic activity and block the sebum secretion.

Up to today, extensive studies have been conducted on lactoferrin. Based on those findings, lactoferrin has become known to have many activities as follows: it deprives Staphylococcus and Escherichia coli of iron by the chelate action and suppresses bacterial and viral proliferation; it increases good bacteria such as acidophilus and bifidobacterium.; it enhances iron intake and prevents anemia; it activates immunocytes such as natural killer cells, and boosts up immunity; it alleviates menstrual cramps, prevents osteoporosis and calms nerves; it works on T-cells to reduce IgE antibody, decreases the histamine production from mast cells, and relieves allergic symptoms such as hay fever; it alleviates stomatitis, bad breath, athlete's foot, etc.; it elevates the level of serum “interleukin-18” against hepatitis C; it protects mucous membranes and alleviates conditions such as dry eye and dry mouse; it controls the production of carcinogenic substances and prevents cancers such as liver cancer and colonic cancer; it suppresses proliferation of protozoan such as toxoplasma, etc.; and it reduces visceral fats.

In regard to the safety of lactoferrin, sufficient data is not available at present. Some advise pregnant women and nursing mothers not to overdose it, but lactoferrin is a component present in breast milk and cow's milk in a large amount and considered to be substantially free of adverse effects to the human body.

Further, concerning the types of lactoferrin, bovine lactoferrin is found in cow's milk and human lactoferrin is found in the human body, and they are thus of different types, however, the consumption of cow's lactoferrin by a person increases human lactoferrin in the blood of the person. This mechanism is yet to be discovered. Lactoferrin is relatively resistant to digestive enzymes and difficult to decompose, but lactoferricin, which has stronger antibacterial activity, is produced from long fragments of lactoferrin decomposed by digestive enzymes. This is presumed to be involved in the foregoing mechanism.

Specific examples of these studies on lactoferrin include the following literatures.

Patent Literature 1 describes acne treatment and claims “use of whey protein fraction comprising lactoferrin to prepare an oral composition for treating an acne.” However, there is no detailed description regarding the action mechanism.

Patent Literatures 2, 3, 4 and 5 relate to treatment of acne exerted by antiinflammation, sebum regulation, antibacterial activity and enhanced β-defensin (antibacterial peptide) production. Patent Literature 3 describes that “hydrolyzed milk protein has been known to be used to alleviate the conditions of hair and skin. This substance can be used as a sebum regulator which improves sebum flow for dry skin and reduces excessive sebum production found in oily skin.” However, lactoferrin is not described. Patent Literature 6 describes anti hair loss effect rendered by the antioxidation activity of lactoferrin and the adverse effects on the hair follicle caused by oxidation stress produced on the scalp surface is reduced, however, the anti-androgenic activity of lactoferrin is not described. Patent Literature 7 is regarding the alleviation of frequent urination and prostatic hypertrophy provided by the antiinflammatory activity of lactoferrin and describes that the inflammation compressing the urinary tract is relieved, making urination easy, however, the anti-androgenic activity is not described. Non Patent Literature 1 mentions the effect of lactoferrin on the sebum but lacks specific disclosures on the details.

Thus, the conventional art discloses the acne treatment (orally), alleviation effect on seborrhiasis (by application), anti hair loss (by application), frequent urination (orally) of lactoferrin or acne treatment by the sebum regulation using hydrolyzed milk protein, however, no literature mentions the anti-androgenic activity of lactoferrin from the aspect of action mechanism thereof.

### CITATION LIST

### Patent Literature

PTL 1: Japanese Patent Application Laid-Open No. 2008-533134
PTL 2: Japanese Patent Application Laid-Open No. 2008-501772
PTL 3: Japanese Patent Application Laid-Open No. 2007-523137
PTL 4: Japanese Patent Application Laid-Open No. 2003-89629
PTL 5: International Publication No. WO 2005-077349
PTL 6: Japanese Patent Application Laid-Open No. 2007-22923
PTL 7: Japanese Patent Application Laid-Open No. 2006-206547

### Non Patent Literature

NPL 1: “Supporting healthy complexion from the inside with Praventin”, DMV international

### SUMMARY OF INVENTION

### Technical Problem

The object of the present invention is to provide an anti-androgenic agent having a strong anti-androgenic activity, free of side effects and very safe, and a sebum secretion blocker and a hair growth stimulant containing the anti-androgenic agent as an active ingredient.

### Solution to Problem

To solve the above problems, some of the natural materials were screened and it has been found that the lactoferrin present in various secretory fluids, such as milk, tears, saliva, bile, and the like, of mammals has a strong anti-androgenic activity, whereby the present invention has been accomplished.

The present invention relates to an anti-androgenic agent comprising lactoferrin.

The present invention relates to a sebum secretion blocker containing the above anti-androgenic agent as an active ingredient.

The present invention relates to a hair growth stimulant containing the above anti-androgenic agent as an active ingredient.

The present invention relates to a food or drink product containing an anti-androgenic agent containing lactoferrin as an active ingredient.

### Advantageous Effects of Invention

The present invention can be used or consumed in everyday life in the form of preparations such as gargles, inhalants and troches, or in the form of snacks such as chewing gums, candies, tablet candies, gummies/jellies, biscuits and chocolates, or food products such as sorbets and beverages; and is useful for preventing or treating hair disorders such as male pattern baldness and coarse hair, diseases such as prostatic hypertrophy and prostate gland tumor, skin diseases such as acne vulgaris and seborrhea caused by excessive sebum secretion, or the like. Further, the anti-androgenic agent of the present invention is very active, free from adverse effects and very safe.

### BRIEF DESCRIPTION OF DRAWINGS

FIG. 1 is a graph showing testosterone 5α-reductase blocking activity.

FIG. 2 is a graph showing blocking rate androgen receptor binding.

FIG. 3 is a graph showing sebum synthesis amount in sebocytes.

FIG. 4 shows micrographic images of Oil Red O staining (results of sebocytes culture test) in sebocytes.

### DESCRIPTION OF EMBODIMENTS

### Example 1

### Testosterone 5α-Reductase Blocking Test

### <Test Method>

1. 0.5 ml of 5 mM Tris-HCl buffer solution (pH 7.2) was added to 0.1 ml of 3.0 mM testosterone solution (dissolved in propylene glycol).

2. Further, 0.1 ml of 6.7 mM NADPH solution (dissolved in 5 mM Tris-HCl buffer solution (pH 7.2)) and 0.05 ml of a sample solution (dissolved in 50% ethanol) were added thereto, the mixture was preheated at 37° C. and 0.1 ml of an enzyme solution (S-9, Oriental Yeast Co., Ltd.) was added to incubate for 1 hour.

In this test, cow's lactoferrin was prepared to be used as the lactoferrin, and saw palmetto was used as a control.

3. Then, 3 ml of dichloromethane was added to terminate the reaction, 0.5 ml of an internal standard solution (0.1 mg/ml of p-hydroxybenzoic acid n-hexyl ester) was added thereto followed by shaking for 10 minutes, and the mixture was centrifuged at 3000 rpm for 10 minutes.

4. The dichloromethane layer was separated, collected and dried under reduced pressure. 5 ml of methanol was added to dissolve the dried product and the solution was used as a sample solution. The residual testosterone was measured by HPLC from the obtained sample solution.

The measurement was carried out by eluting with methanol/water (65/35) and monitoring at 254 nm under the conditions of using a YMC A-302 (internal diameter 4.6×150 mm) column at a column temperature of 40° C. at a flow rate of 1 ml/min. The measurement was carried out using the internal standard substance method and the blocking rate (%) was determined using the following formula.

blocking rate(%)=(A−B)/(C−B)×100

A: Testosterone amount when the test solution was added

B: Testosterone amount with a control for 30 minutes (testosterone amount when the reaction was carried out using a 50% ethanol solution in place of the sample solution)

C: Testosterone amount with a control for 0 minute (testosterone amount when dichloromethane was added after mixing the Tris-hydrochloric acid buffer solution, testosterone, the sample solution and the enzyme solution and before adding NADPH so as not the reaction to proceed.)

### <Test Result>

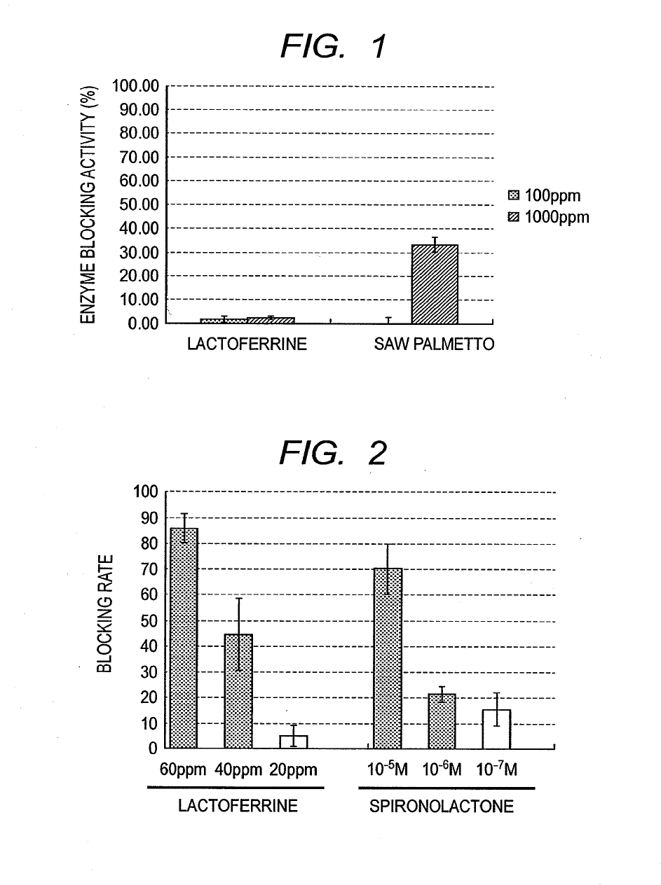

FIG. 1 shows the obtained results. As evident in FIG. 1, saw palmetto, the positive control, showed a blocking activity of about 35% at a concentration of 1000 ppm, whereas lactoferrin showed no testosterone 5α-reductase blocking activity.

### Example 2

### Androgen Receptor Binding Blocking Test

### <Test Method>

1. An androgen-dependent mouse breast cancer cell SC-3 cell was inoculated in a 96-microplate using 2% DCC treated FCS-containing MEM medium at a cell density of 1.0×10⁴ cells/well/100 μl and cultured at 37° C. under conditions of 5% CO₂-95% air.

2. 24 Hours later, the culture medium was exchanged with HMB medium (0.1% BSA-containing Ham F12+MEM medium) to which the test sample and 10⁻⁸M DHT were added, and the cell was cultured for 48 hours.

In this test, cow's lactoferrin was prepared to be used as the lactoferrin, and spironolactone was used as a positive control.

3. Then, in accordance with MTT assay, the cell proliferation was evaluated by measuring the absorbance at 570 nm, which is the absorption maximum of blue formazan.

To correct the influence of adherent cells, the absorbance at 650 nm was also measured simultaneously, and the difference between both absorbances was used as the produced amount of blue formazan. At the same time with the above, to examine the effect of the sample alone on SC-3 cell, the same culture and measurement were carried out by adding the sample alone, without adding DHT, to the HMB medium. Further, as controls, the same measurement was carried out for the case wherein the cell was cultured in HMB medium to which the sample and DHT were not added and for the case wherein the cell was cultured in HMB medium to which DHT alone was added but not the sample. Based on the measurement results, the binding blocking rate representing the anti-androgenic activity was determined using the following formula.

Binding blocking rate(%)=[{(A−B)−(C−D)}/(A−B)]×100

A: The absorbance when DHT was added but the sample was not added

B: The absorbance when DHT was not added and the sample was not added

C: The absorbance when DHT was added and the sample was added

D: The absorbance when DHT was not added but the sample was added

### <Test Result>

FIG. 2 shows the obtained results. As evident in FIG. 2, spironolactone, the positive control, showed a binding blocking activity of about 70% at a concentration of 10⁻⁵M. Lactoferrin also had concentration-dependent binding blocking activity and showed a blocking activity of about 85% at a concentration of 60 ppm.

### Example 3

### Sebocyte Culture Test

### <Test Method>

1. The test was carried out using a hamster sebocyte culture kit KB-1000 (manufactured by KURABO INDUSTRIES LTD.).

The sebocyte cell derived from the auricula of a normal golden hamster was inoculated in a 24-well plate in a density of 5.0×10⁴ cells/well. During the test, the cell was cultured in medium containing testosterone in a concentration of 1×10⁻⁶M.

2. The cell was cultured for several days until confluent, and the medium was exchanged with medium containing the test sample. The medium was exchanged with sample medium every other day and the culture was continued for about 2 weeks.

In this test, cow's lactoferrin was prepared to be used as the lactoferrin, and spironolactone was used as a control.

3. Then, using a sebum synthesis assay kit SE-3001 (manufactured by KURABO INDUSTRIES, LTD.), a WST-8 solution was added to each well and incubated for 30 minutes at 37° C. The supernatant was assayed at a wavelength of 450 nm, and the number of viable cell was counted.

4. Further, the supernatant was stained with Oil Red O and observed under a microscope while extracted with 100% isopropanol and measured at a wavelength of 520 nm for the lipid synthesis amount.

The correction was made using the following formula, and the lipid synthesis amount per cell was compared.

Comparison of lipid synthesis amount per cell=absorbance B/absorbance A

Absorbance A: cell number measured value (450 nm)

Absorbance B: lipid synthesis amount measured value (520 nm)

### <Test Result>

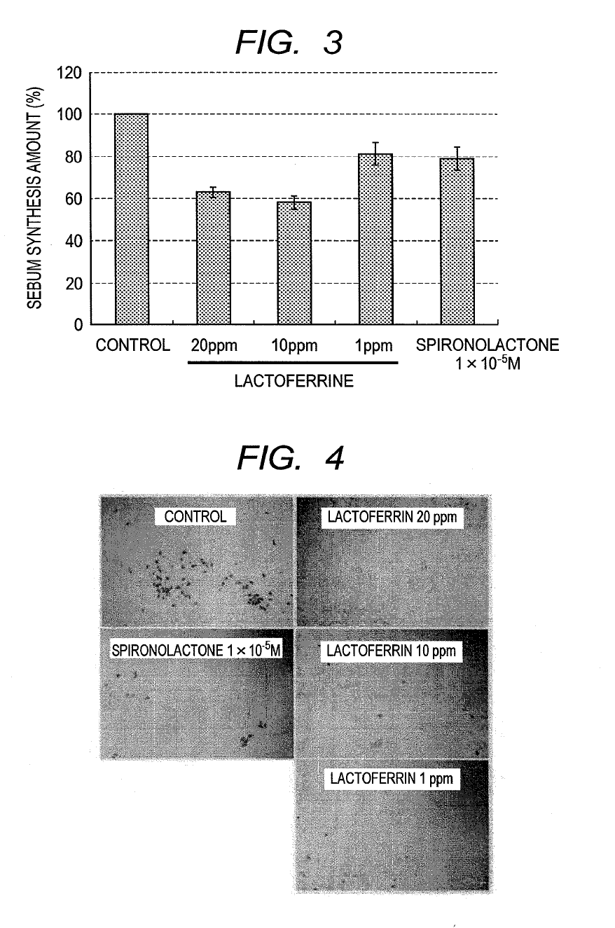

FIG. 3 and FIG. 4 show the obtained results. As evident in FIG. 3 and FIG. 4, spironolactone, the positive control, had a decreased sebum synthesis amount compared with the control at a concentration of 1×10⁻⁵ M in the microscopic observation and the absorbance measurement. However, lactoferrin, in the microscopic observation and the absorbance measurement, suppressed the sebum synthesis amount in a concentration-dependent manner and suppressed the sebum synthesis amount to 60% at concentrations of 10 ppm and 20 ppm.

Next, using lactoferrin, tablet pharmaceutical, chewing gum, candy, chocolate, biscuit, gummy jelly, tablet candy, tablet confectionary, ice cream, sorbet, and beverage were prepared by the routine method. The formulations are shown below. The product range of the present invention is not limited to these products.

### Example 4

A tablet pharmaceutical was prepared in accordance with the following formulation.

| | |
|---|---|
| D-mannitol | 42.6% |
| Lactose | 42.6 |
| Crystalline cellulose | 8.5 |
| Hydroxypropylcellulose | 4.3 |
| Lactoferrin | 2.0 |
| | 100.0% |

### Example 5

A chewing gum was prepared in accordance with the following formulation.

| | |
|---|---|
| Gum base | 20.0% |
| Sugar | 54.7 |
| Glucose | 14.5 |
| Starch syrup | 9.3 |
| Flavor | 0.5 |
| Lactoferrin | 1.0 |
| | 100.0% |

### Example 6

A candy was prepared in accordance with the following formulation.

| | |
|---|---|
| Sugar | 50.0% |
| Starch syrup | 31.4 |
| Citric acid | 1.0 |
| Flavor | 0.2 |
| L-menthol | 1.0 |
| Lactoferrin | 2.0 |
| Water | 14.4 |
| | 100.0% |

### Example 7

A chocolate was prepared in accordance with the following formulation.

| | |
|---|---|
| Cacao bitter | 20.0% |
| Whole milk powder | 20.0 |
| Cacao butter | 17.0 |
| Powdered sugar | 40.45 |
| Lecithin | 0.45 |
| Flavor | 0.1 |
| Lactoferrin | 2.0 |
| | 100.0% |

### Example 8

A biscuit was prepared in accordance with the following formulation.

| | |
|---|---|
| Sugar | 31.7% |
| Flour | 26.8 |
| Cornstarch | 26.8 |
| Butter | 3.2 |
| Egg | 10.2 |
| Sodium bicarbonate | 0.3 |
| Lactoferrin | 1.0 |
| | 100.0% |

### Example 9

A gummy jelly was prepared in accordance with the following formulation.

| | |
|---|---|
| Polydextrose aqueous solution | 39.0% |
| Sorbitol aqueous solution | 8.0 |
| Paratinose aqueous solution | 9.0 |
| Maltose aqueous solution | 20.0 |
| Trehalose aqueous solution | 11.0 |
| Gelatin | 10.0 |
| Tartaric acid | 1.0 |
| Lactoferrin | 2.0 |
| | 100.0% |

### Example 10

A tablet candy was prepared in accordance with the following formulation.

| | |
|---|---|
| Sugar | 46.65% |
| Glucose | 19.0 |
| Sucrose fatty acid ester | 0.2 |
| Flavor | 0.15 |
| Lactoferrin | 30.0 |
| Water | 4.0 |
| | 100.0% |

### Example 11

A tablet confectionary was prepared in accordance with the following formulation.

| | |
|---|---|
| Sugar | 35.85% |
| Sucrose fatty acid ester | 0.15 |
| Lactoferrin | 60.0 |
| Water | 4.0 |
| | 100.0% |

### Example 12

An ice cream was prepared in accordance with the following formulation.

| | |
|---|---|
| Yolk | 11.0% |
| Sugar | 14.0 |
| Cow's milk | 35.5 |
| Whipped cream | 37.0 |
| Vanilla bean | 0.5 |
| Lactoferrin | 2.0 |
| | 100.0% |

### Example 13

A sorbet was prepared in accordance with the following formulation.

| | |
|---|---|
| Orange fruit juice | 16.0% |
| Sugar | 31.0 |
| Lactoferrin | 3.0 |
| Water | 50.0 |
| | 100.0% |

### Example 14

A beverage was prepared in accordance with the following formulation.

| | |
|---|---|
| Orange fruit juice | 30.0% |
| Isomerized sugar syrup | 13.34 |
| Citric acid | 0.1 |
| Vitamin C | 0.04 |
| Flavor | 0.1 |
| Lactoferrin | 2.0 |
| Water | 54.42 |
| | 100.0% |

### INDUSTRIAL APPLICABILITY

The present invention is useful as an additive material to a range of products containing lactoferrin having an anti-androgenic activity as an active ingredient. The present invention is also applicable to products such as candies and gums, and new health functional products.

This application claims the priority to the Japanese patent Application No. 2009-292651, filed on Dec. 24, 2009, which is hereby incorporated by reference as a part of this application.

1-4. (canceled)

5. A method of hair growth stimulation, comprising orally administering an antiandrogen agent including lactoferrin.

6. A method of hair growth stimulation, comprising ingesting a food or drink product including an antiandrogen agent containing lactoferrin.

7. A method of sebum secretion inhibition, comprising orally administering an antiandrogen agent including lactoferrin.

8. A method of sebum secretion inhibition, comprising ingesting a food or drink product including an antiandrogen agent containing lactoferrin.
